(19) United States
(12) Patent Application Publication
(10) Pub. No.: US 2018/0333338 A1
(43) Pub. Date: Nov. 22, 2018

(54) SLOSH-DISINTEGRATING CLEANSING WIPES

(71) Applicant: Medline Industries, Inc., Northfield, IL (US)

(72) Inventors: Alana Cecola, Round Lake, IL (US); Sharbel Maalouf, Pleasant Prairie, WI (US); Sohum Mansukhani, Chicago, IL (US)

(21) Appl. No.: 15/962,669

(22) Filed: Apr. 25, 2018

Related U.S. Application Data
(60) Provisional application No. 62/507,871, filed on May 18, 2017.

(51) Int. Cl. A61K 8/02; A61Q 19/10; A61F 13/15

(52) U.S. Cl. CPC A61K 8/0208; A45D 2200/1027; A61F 13/15211; A61Q 19/10

(57) ABSTRACT

Disclosed are bathing wipes that comprise a flushable material impregnated with a non-rinsing cleansing solution. The wipes are suitable for use in cleansing a bedridden patient in accordance with a specified bathing protocol. To use the package of wipes, one or more caregivers removes one wipe to bath the patient in accordance with the bathing protocol, then discards the wipe, optionally by flushing the wipe into a conventional sanitary system. Also disclosed are methods for bathing a patient.

### TECHNICAL FIELD

The disclosure is in the field of patient care, and more particularly relates to bathing of patients by caregivers.

### BACKGROUND

Hospitals and other care facilities must periodically bathe bedridden patients. A typical hospital protocol will provide for a specified number of patient baths for a given time period (for example, five baths per week). It is known to provide packages of bathing wipes for use in accordance with such protocol. For instance, the wipes might be sold in a package of five, corresponding to one week of bathing under the specified bathing protocol. The wipes also can be sold in a package that contains a number of wipes not corresponding to a predetermined bathing protocol but packaged in a convenient number, for example, a package of five or eight wipes.

Conventional bating wipes are composed of a soft, nonwoven material that is impregnated with a cleansing solution. Typically, the cleansing solution is a non-rinsing solution, by which is contemplated that the cleansing solution may be applied to the patient without the need to subsequently rinse the cleansing solution off of the patient with water, as would be impractical for a bedridden patient. Typical such cleansing solutions include moisturizers and other components intended to ensure compatibility with patient skin. After bathing the patient with a wipe, the wipe is discarded.

It has now been discovered that a bathing wipe can be made of a slosh-disintegrating, flushable material. Flushability may be assessed in accordance with one or more industry guidelines. The wipes may be impregnated with a cleansing solution, such as a typical cleansing solution known in the art and may be packaged with a number of wipes corresponding to a predetermined bathing protocol or otherwise as may be found to be suitable or convenient. After use, a wipe may be discarded by flushing the wipe into a conventional sanitary system. The wipe will disintegrate upon sloshing in a conventional toilet sufficiently so as not to clog the sanitary system, and will be sufficiently biodegradable to be compatible with conventional sewerage systems.

### BRIEF DESCRIPTION OF THE DRAWINGS

FIG. 1 illustrates one wipe in accordance with the present teachings.

FIG. 2 illustrates a package of wipes in accordance with the present teachings.

### DETAILED DESCRIPTION

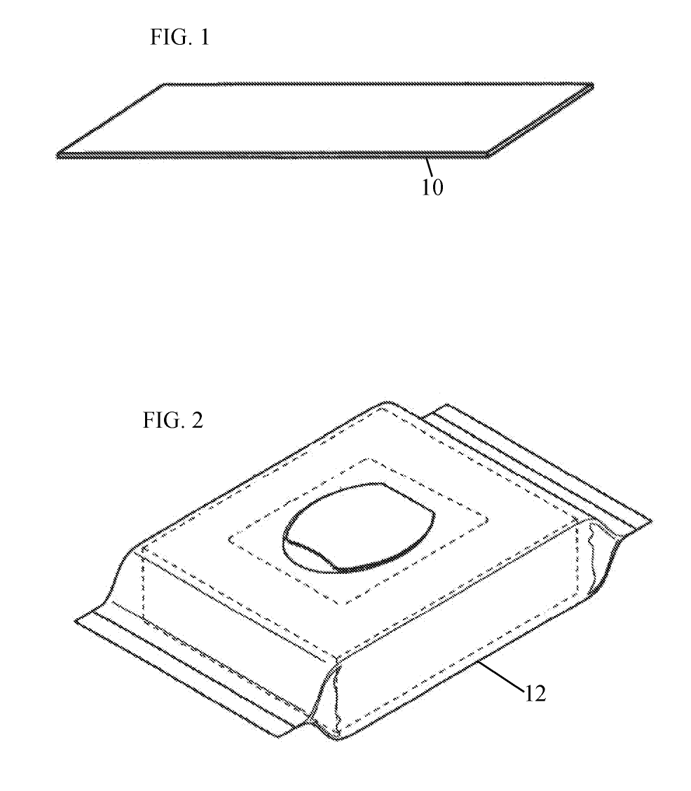

With reference to FIG. 1, wipe 10 may be provided in any suitable size and shape for a bathing wipe. The wipe is typically approximately square in cross section and may be of any suitable size, such as 8 inches×8 inches in cross section. The package of wipes 12 shown in FIG. 2 may comprise wipes folded and stacked in a conventional matter and disposed is then the conventional package, for example, a foil or film package. Package 12, as illustrated, contains eight wipes.

The wipes should be slosh-disintegrating wipes, by which is contemplated that the wipes may be flushed into a conventional sanitary system in a manner that that wipes will disintegrate sufficiently so as not to clog or otherwise damage the sanitary system. Conventional sanitary systems include at least a 3 inch diameter sanitary drain pipe and have a minimum slope of ¼ inch per foot, and drain into a sewerage system, although in some cases the drainage is to a septic area.

The flushability may be assessed via any suitable manner. For instance, flushability may be assessed in accordance with standards FG501 through FG507 of the Association of the Nonwoven Fabrics Industry of Cary, N.C. To qualify as “flushable” under these standards, each of the tests FG501 through FG507 should be satisfied.

The wipes may be impregnated with any suitable cleansing composition. Preferably, the cleansing composition is a non-rinsing cleansing composition that is suitable for bathing a bedridden patient and does not require application of water to rinse the cleansing composition after bathing. The cleansing composition may be provided with a fragrance if desired, or a fragrance may be omitted. Preferably, the cleansing composition does not include an antimicrobial material, to ensure biodegradability and compatibility with conventional sanitation systems, although in some cases an antimicrobial compound may be included. An exemplary cleansing composition is provided below:

| Ingredient | % w/w |
|---|---|
| Water | 98.523 |
| Linoleamidopropyl PG-Dimonium Chloride | 0.3 |
| Phosphate | |
| Myristamidopropyl PG-Dimonium Chloride | 0.3 |
| Phosphate | |
| Polysorbate 20 | 0.1 |
| Phenoxyethanol | 0.05 |
| Disodium EDTA | 0.5 |
| Benzalkonium Chloride | 0.5 |
| Aloe Barbadensis Leaf Juice | 0.002 |
| Citric Acid | 0.045 |
| Dow Corning 1520 Silicon Emulsion | 0.1 |

The wipe may be composed of any suitable nonwoven fabric substrate. One suitable material is sold commercially as NBOND Non-Woven by the Hangzhou NBond Non-Woven Company, Ltd. The NBond material is a cellulosic material composed of wood pulp, and has been found to meet the heretofore referenced standards FG501 through FG507. Each wipe may have any suitable basis weight, for example, 60-80 GSM basis weight. Any other suitable flushable substrate may be employed.

In use, the method of bathing a patient wipes may be as simple as opening the package, removing a wipe, and using the wipe to bath a patient, and subsequently discarding the wipes via flushing or otherwise. The method is contemplated to be mostly useful in bathing of patients by a caregiver, although in some cases the wipes may be used by a person to bathe himself or herself. In some instances, the wipes are packaged to correspond to a single period of use with a predetermined number of baths per period in accordance with a cleansing protocol (for example, a package of five wipes corresponding to five baths per week). In this case, the method of using the package comprises following the patient bathing protocol in the given period by using one wipe per bath to bathe the patient. After each bath, the wipes are discarded, optionally by flushing. It is contemplated that the wipes need not be disposed of by flushing and might instead be disposed of conventionally.

The following example is provided to illustrate the present application.

### Example

A 70 GSM sample of NBond spun bonded material is impregnated with a cleansing composition having the exemplary cleansing composition described above. The sample is composed of 8 inch×8 inch wipes. The wipes are folded into a conventional stacked arrangement and sealed within a polymeric outer packaging material.

All references cited herein are hereby incorporated by reference in their entireties.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A package containing plural cleansing wipes, each of said wipes being impregnated with a non-rinsing cleansing solution, each of said wipes comprising a flushable nonwoven wipe.

2. A package according to claim 1, the package containing between five and eight wipes.

3. A package according to claim 1, containing eight wipes.

4. A package according to claim 1, each of said wipes having a basis weight of from 60-80 GSM.

5. A package according to claim 1, each of said wipes comprising an approximately square cross section.

6. A method for bathing a patient, comprising
providing a bathing protocol specifying multiple bathing sessions per time period;
providing a package of plural wipes, each of said wipe being impregnated with a non-rinsing cleansing solution, each of said wipes comprising a flushable nonwoven wipe, the number of wipes in said package corresponding to the number of bathing sessions in said time period; and
bathing said patient with one wipe per bathing session in accordance with said bathing protocol.

7. A method according to claim 6, said package containing between five and eight wipes.

8. A method according to claim 6, said package containing eight wipes.

9. A method according to claim 6, each of said wipes having a basis weight of from 60-80 GSM.

10. A method according to claim 6, each of said wipes comprising an approximately square cross section.

11. A method comprising:
providing a package of wipes being impregnated with a non-rinsing cleansing solution, each of said wipes comprising a flushable nonwoven wipe;
removing a wipe from said package;
bathing a patient with said wipe; and
discarding said wipe.

12. A method according to claim 11, comprising discarded said wipe via flushing said wipe into a sanitary system.
